(19) United States
(12) Patent Application Publication
(10) Pub. No.: US 2010/0278688 A1
(43) Pub. Date: Nov. 4, 2010

(54) Containerized Medical Waste Treatment System and Related Method

(76) Inventors: Damien Tanaka, Redding, CT (US); Paul Cramer, Bristol, CT (US)

Correspondence Address: MCCARTER & ENGLISH, LLP HARTFORD;CITYPLACE I, 185 ASYLUM STREET, HARTFORD, CT, 06103, US

(21) Appl. No.: 12/625,378

(22) Filed: Nov. 24, 2009

Related U.S. Application Data
(60) Provisional application No. 61/117,573, filed on Nov. 24, 2008.

(51) Int. Cl. A61L 2/07; A61L 2/04; A61L 2/26; A61L 2/24; B65G 47/00; B65G 41/00

(52) U.S. Cl. 422/26; 422/297; 422/291; 422/119; 422/309

(57) ABSTRACT

A containerized medical waste treatment system for sterilizing and processing waste. The system includes an elongate, substantially enclosed container-like structure with an access door on at least one lateral side to accommodate the entry of moveable carts containing medical waste. The system further includes an autoclave for sterilizing medical waste, a shredder for processing the load after sterilization, a tipper for transferring the waste held in carts into the shredder, and a moveable roof panel for providing clearance for a cart that has been elevated by the tipper. At least one conveyor belt for receiving and directing sterilized, shredded waste into a previously positioned waste container is included. The system can perform an autoclave sterilization method which includes relatively short evacuation and heating cycles to remove air pockets within the mass of the load to reduce autoclave treatment times.

### CROSS-REFERENCE TO RELATED APPLICATION

This application claims priority to similarly titled U.S. Patent Application No. 61/117,573, filed Nov. 24, 2008, the contents of which is expressly incorporated by reference in its entirety as part of the present disclosure.

### FIELD OF THE INVENTION

The present disclosure generally relates to the field of waste disposal, and more particularly, to containerized systems and methods for providing medical waste processing.

### BACKGROUND OF THE INVENTION

The term “medical waste” is a generic term commonly used to describe medical infectious and non-infectious waste, including waste categories such as (1) cultures and stocks of infectious agents and associated biologicals; (2) pathological wastes; (3) human blood and blood products; (4) contaminated “sharps”, including needles, syringes, blades, scalpels, and broken glass; (5) animal waste; (6) isolation waste, including gloves and other disposable products used in the care of patients; and (7) unused “sharps”.

It has been reported that the amount of waste generated in U.S. hospitals is approximately 6,670 tons per day, or about 1% of the 158 million tons of municipal solid waste produced annually. It has been further reported that 15% of the total waste generated by hospitals (over 1,000 tons daily) can be regarded as medical waste. While hospitals can be considered the primary medical waste producer, the aforementioned figures capture only a fraction of the healthcare facilities that generate medical waste. For example, currently there are approximately 180,000 private physicians' offices, 98,000 private dentists' offices, 38,000 veterinarians'offices, 15,000 medical clinics, 12,000 long-term care facilities, 4,000 laboratories, and 900 free-standing blood banks. The proper disposal of these facilities' waste is of particular concern because medical waste may be contaminated with dangerous microorganisms of infectious diseases. As such, medical waste must be sterilized before disposal in a landfill, for example.

In the past, individual facilities have handled the sterilization and/or processing of their medical waste with on-site systems. Thus, each facility dedicated valuable square-footage in housing the processing equipment and other related areas throughout the facility, such as storage areas, loading areas, transportation areas, and collection areas. Also, these individual facilities incurred great expense in the construction, maintenance, repair and operation of the processing equipment and in the staffing associated with performing the treatment processes. Facilities without on-site medical waste treatment systems also incurred great cost related to the hauling away, processing, sterilization and/or disposal of their medical waste.

One piece of equipment commonly used by both on-site and off-site waste processing systems is an autoclave. An autoclave is a device that uses steam to sterilize medical waste and other objects prior to disposal in the standard municipal solid waste stream. Autoclaving has grown as an alternative to incineration due to environmental and health concerns raised by combustion byproducts from incinerators, especially from the small units which were commonly operated at individual medical facilities. The terms “autoclave” and “autoclaving” are used to describe a machine or process in which elevated temperature and pressure are used in processing unsterile materials into sterile materials.

As the goal of autoclaving is to achieve sterility, it is very important to ensure that all of the air in the autoclave vessel is removed. Hot air is very poor at achieving sterility as compared to steam. Steam at 134° C. can achieve in 3 minutes the same sterility that hot air at 160° C. can take two hours to achieve. Prior art autoclaves and autoclaving methods achieve air removal by various means including downward displacement (or gravity type), steam pulsing, vacuum pumps, superatmospheric cycles, and subatmospheric cycles. However, prior art systems and methods have not sufficiently accounted for air pockets that tend to form within the mass of objects being autoclaved. An air pocket located within a mass may prevent the interior of the mass from reaching sterilizing temperatures as quickly as the remainder and/or exterior of the mass, and thus some prior art systems and methods require more time and energy to complete sterilization than is otherwise desired.

Accordingly, it is an object of the present disclosure to overcome one or more of the above-described drawbacks and/or disadvantages of the prior art.

### SUMMARY OF THE INVENTION

In accordance with a first aspect, the present disclosure is directed to a containerized medical waste treatment system (hereinafter “containerized treatment system”). The containerized treatment system allows, if desired, for a mobile unit which can perform a complete waste handling and sterilization process. The containerized treatment system can function as a less expensive alternative to either transporting medical waste to a remote sterilization center, or to an on-site medical waste treatment system. In a currently preferred embodiment of the present disclosure, the containerized treatment system can be placed at a loading dock of a facility, accept carts of waste from the facility either from the dock or from ground level, sterilize the waste in an autoclave adapted to receive the carts of waste, mechanically/hydraulically tip a cart of sterilized waste from the autoclave to transfer the sterilized waste into a shredder, shred the sterilized waste into relatively small unrecognizable pieces, and transfer the sterilized, shredded waste by a conveyor, such as a two-belt conveyor system, to a nearby refuse container.

In accordance with another aspect, the present disclosure is directed to a containerized waste treatment system for sterilizing and processing waste. The system comprises an elongate enclosure defining a front end, a rear end, and at least two side walls laterally spaced on substantially opposite sides of the enclosure relative to each other and extending between the front and rear ends of the enclosure. A platform extends in an elongate direction between the front and rear ends, and extends laterally between the two side walls of the enclosure. An access opening, such as a side door, extends through a side wall of the enclosure, or if desired, each side wall includes an access opening, to allow entry of an operator and cart into the enclosure and egress out of the enclosure therethrough. An autoclave is mounted within the enclosure on the platform adjacent to either the front end or the rear end of the enclosure. A shredder is mounted within the enclosure on the platform adjacent to either the front end or the rear end of the enclosure. The shredder and the autoclave are spaced relative to each other and define a cart path therebetween within the enclosure. The cart path is in communication with the access opening to allow the operator to move a cart containing a load of waste through the access opening, onto the cart path, from the cart path into the autoclave, sterilize the load of waste in the autoclave, retrieve the load of waste from the autoclave, move the cart with the sterilized load of waste along the cart path to the shredder, move the load of sterilized waste from the cart into the shredder, shred the sterilized waste in the shredder, and dispose of the sterilized, shredded waste.

In some currently preferred embodiments, the containerized treatment system comprises (i) a standard semi-truck trailer or a standard semi-truck shipping container; (ii) at least one side door located at about the midpoint of the enclosure for introducing a cart of waste into the enclosure; (iii) an autoclave mounted within the enclosure forwardly of the at least one side door and approximately over support structure(s) for receiving the waste from the cart and sterilizing the waste by the application of steam; (iv) a shredder for shredding sterilized waste located rearwardly of the at least one side door and approximately over the rear wheels of the enclosure or of a platform beneath the enclosure; (v) a cart track extending from the floor of the enclosure to the autoclave for loading carts of waste into the autoclave; (vi) a tipper located forwardly of the shredder for transferring the contents of the cart into the shredder and, in turn, shredding the waste; (vii) a moveable roof panel located on the roof of the enclosure above the tipper or shredder to allow for increased vertical space to accommodate the cart when lifted by the tipper over the shredder to transfer the waste into the shredder; (viii) a conveyor positioned to accept shredded, sterilized waste from the outlet of the shredder and transport the shredded, sterilized waste to a receptacle, including a lower translatable belt, that is laterally translatable from one side of the enclosure to the other, and an upper stationary belt located at the outlet of the shredder and extending from approximately one side of the enclosure to the other side; (ix) at least one hinged member located on at least one side of the enclosure and extending from or between the rear end of the enclosure to a respective side door, wherein the at least one hinged member can pivot from an open position that is substantially perpendicular to the respective side of the enclosure to a closed position that is substantially parallel to, or otherwise located against or immediately adjacent to the respective side of the enclosure, wherein at least one end of the member is capable of pivoting downwardly in order to contact the ground; and (xi) utility connections mounted on the sides or bottom of the enclosure to provide utilities to the components of the system, including a plurality of electrical connections, a steam connection, a water connection, and a drain connection.

In some embodiments, the containerized treatment system includes a plurality of utility connections located at the facility that correspond to a plurality of the utility connections mounted on the enclosure to provide utilities to the components of the system, such as a plurality of electrical connections, a steam connection, a water connection, and a drain connection. The utility connections may or may not include quick-disconnect type connections. In some embodiments, the containerized treatment system further includes an umbilical cord for connecting utility connections on the enclosure to utility connections at the facility. In another embodiment, the containerized treatment system includes an umbilical connection extending from the enclosure, that houses utility connections, and is capable of providing utilities to the system. In such an embodiment, the system further includes a substantially similar umbilical connection located at the facility and capable of connecting to the umbilical connection extending from the enclosure to thereby provide utilities to the system.

In some embodiments of the present disclosure, the containerized treatment system includes a document destruction and collection device. In a currently preferred embodiment of the present disclosure, the containerized waste treatment system performs paper document destruction with the onboard waste shredder. The documents are loaded into the shredder in a manner that is the same as, or similar to, the manner of loading other waste into the shredder, in that the documents can be loaded through carts and a tipper associated with the shredder. In addition, the containerized treatment system may include means to collect and transport the destroyed paper documents from the outlet of the shredder. The collection means may take the form of a vacuum mounted to apply suction to the top of a conveyor belt which is sized and positioned to receive the outlet of the shredder. In such an embodiment, the vacuum removes the shredded documents from the conveyor belt and transports the material through a conduit to a waste receptacle. In some embodiments of the present disclosure, the shredded documents are transported to a separate waste receptacle, such as paper recycling or other recycling bin, to facilitate recycling of the shredded documents.

In accordance with another aspect, the present disclosure is directed to a sterilization method. The sterilization method comprises the following steps:

(i) providing a containerized enclosure including a side door, an autoclave mounted to one side of the side door within the interior of the enclosure, and a shredder mounted to an opposite side of the side door within the interior of the enclosure, and a cart path extending between the side door, autoclave and shredder;

(ii) transporting the containerized enclosure with a vehicle from a first location to a second location;

(iii) with the enclosure located in the second location, introducing a cart containing a load of waste through the side door into the enclosure;

(iv) placing the cart within the autoclave and sterilizing the load of waste within the autoclave;

(v) moving the cart with the sterilized load of waste along the cart path from the autoclave to the shredder;

(vi) moving the sterilized waste from the cart into the shredder and shredding the sterilized waste; and

(vii) discharging the shredded sterilized waste from the enclosure into a nearby refuse container.

In accordance with another aspect, the present disclosure is directed to a sterilization method including the following steps:

(i) sealing an autoclave to achieve a substantially air-tight space within the autoclave and surrounding a load of waste;

(ii) performing a first evacuation, wherein the first evacuation removes air from the autoclave and brings about a pre-determined first pressure below atmospheric to the load;

(iii) introducing condensable vapor having transferable latent heat at a pre-determined first temperature to the substantially air-tight space, wherein the introduction brings about a pre-determined first pressure above atmospheric to the load;

(iv) allowing the condensable vapor to transfer heat to the load for a first time period under the first pressure above atmospheric;

(v) performing a second evacuation, wherein the second evacuation removes a pre-determined amount of the condensable vapor and air from the autoclave to bring about a second pressure below atmospheric to the load;

(vi) introducing condensable vapor having transferable latent heat at a pre-determined second temperature to the substantially air-tight space, wherein the introduction brings about a pre-determined second pressure above atmospheric to the load;

(vii) allowing the condensable vapor to transfer heat to the load for a second time period under the second pressure above atmospheric, wherein the second time period is substantially the same as or similar to the first time period;

(viii) performing a third evacuation, wherein the third evacuation removes condensable vapor and air from the autoclave to bring about a third pressure below atmospheric to the load;

(ix) introducing condensable vapor having transferable latent heat at a pre-determined third temperature to the substantially air-tight space, wherein the introduction brings about a pre-determined third pressure above atmospheric to the load;

(x) allowing the condensable vapor to transfer heat to the load for a third time period under the third pressure above atmospheric, wherein the third time period is substantially longer as compared to the first and second time periods, to substantially sterilize the load; and

(xi) performing a fourth evacuation, wherein the fourth evacuation removes condensable vapor and air from the autoclave to bring about a fourth pressure below the third pressure.

In some such embodiments, the second pressure below atmospheric is substantially similar to the first pressure below atmospheric; the pre-determined second pressure above atmospheric is substantially similar to the pre-determined first pressure above atmospheric; the third pressure below atmospheric is substantially the same as or similar to the first and second pressures below atmospheric; and the pre-determined third pressure above atmospheric is substantially similar to the pre-determined first and second pressures above atmospheric.

In a currently preferred embodiment, the load is medical waste, the first and second heating time periods are each within the range of about 2 minutes to about 10 minutes, and the heat during at least the third time period is sufficiently high to sterilize the medical waste. In one embodiment, the first, second, or third temperatures are each within the range of about 275 degrees Fahrenheit to about 300 degrees Fahrenheit. In another embodiment, the first, second, and third pressures below atmospheric are each within the range of about 6 inches of water vacuum to about 20 inches of water vacuum. The sterilization method may further include the step of allowing the sterilized medical waste to cool to a handling temperature. In alternative embodiments, steps (v), (vi), and (vii) define a cycle which may be repeated at least one time before step (viii). If an air pocket forms within the mass of the load during such first or second cycles, the method may loosen the mass of the load, or otherwise act to remove the air pocket from the mass, and further remove from the autoclave the air from the air pocket.

One advantage of the containerized medical waste treatment system and method is that they allow for a unit that can be transported, and if desired, may itself be mobile, and which can perform a complete waste handling and sterilization process at, for example, a remote facility. Another advantage of the containerized medical waste treatment system and method is that they can function as a less expensive alternative to transporting medical waste to a remote sterilization center or to an on-site medical waste treatment system. Yet another advantage of some currently preferred embodiments of the containerized medical waste treatment system and method is that they provide a document destruction and recycling process along with medical waste sterilization and processing. This feature can be significantly advantageous with respect to properly shredding and disposing of Health Insurance Portability and Accountability Act (“HIPAA”) documents, for example. Yet another advantage is that providing one system, that performs both document shredding and medical waste disposal functions, can give rise to enhanced efficiencies and significant cost savings in comparison to the prior art. Another advantage of the currently preferred embodiment of the sterilization method is that the method allows for shorter overall sterilization cycle times as compared to prior art autoclave systems.

These and other advantages of the present invention, and/or of the currently preferred embodiments thereof, will become more readily apparent in view of the following detailed description of the currently preferred embodiments and the accompanying drawings.

### BRIEF DESCRIPTION OF THE DRAWINGS

FIG. 1 is a top view of a currently preferred embodiment of a containerized medical waste treatment system of the present disclosure with the top wall removed;

FIG. 2 is a left side view of the containerized medical waste treatment system of FIG. 1 with the left wall removed;

FIG. 3 is a right side view of the containerized medical waste treatment system of FIG. 1 with the right wall removed;

FIG. 4a is a front side view of a currently preferred embodiment of a medical waste sterilization cart of the present disclosure;

FIG. 4b is a bottom view of the medical waste sterilization cart of FIG. 4a;

FIG. 4c is a right side view of the medical waste sterilization cart of FIG. 4a;

FIG. 5 illustrates front, perspective, and side views, respectively, of exemplary electrical connections of the containerized medical waste treatment system of FIG. 1; and

FIG. 6 illustrates front, perspective, and side views, respectively, of exemplary non-electrical utility connections of the containerized medical waste treatment system of FIG. 1.

### DETAILED DESCRIPTION OF PREFERRED EMBODIMENTS

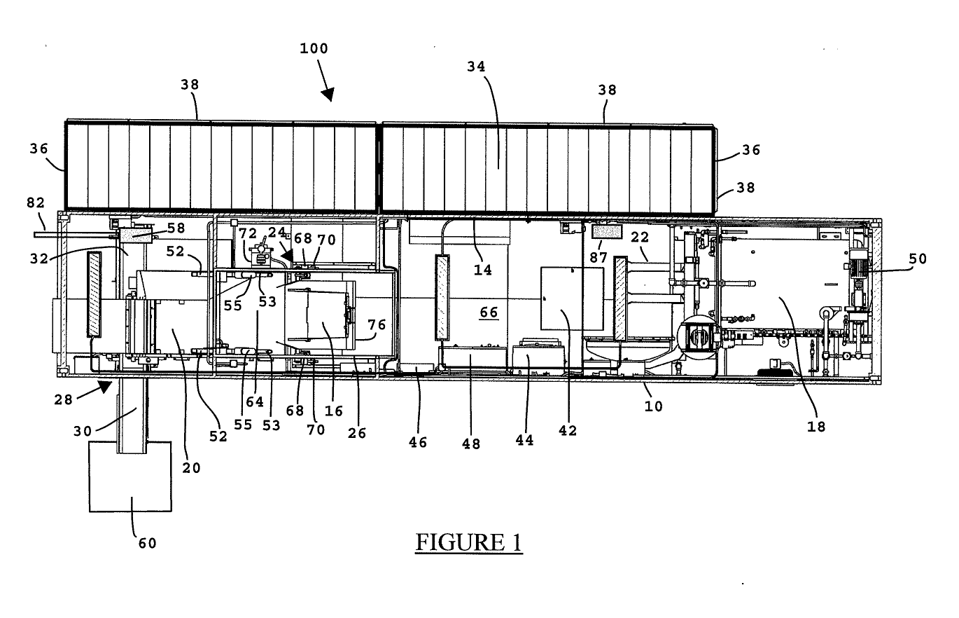

In FIGS. 1-3, a containerized medical waste treatment system (hereinafter “containerized treatment system”) is indicated generally by the reference number 100. As is shown in FIGS. 1-3, the containerized treatment system can perform a complete waste handling and sterilization process. The containerized treatment system 100 can function as a less expensive alternative to either transporting medical waste to a remote sterilization center or an on-site medical waste treatment system where the system is permanently installed within a facility. The containerized treatment system 100 can be placed along a pre-existing loading dock of a facility, accept carts of waste from the facility either from the dock or from ground level, sterilize the waste in an autoclave designed to receive the carts of waste, mechanically/hydraulically tip a cart of sterilized waste from the autoclave to transfer the sterilized waste into a shredder, shred the sterilized waste into relatively small unrecognizable pieces, and transfer the sterilized, shredded waste by a two-belt conveyor system to a previously positioned refuse container.

The main structure containing the sterilization and processing equipment is a container or enclosure 10. The enclosure 10 houses the sterilization and processing equipment and provides a base that permits them to be operated and transported from one location to another. In the illustrated embodiment, the enclosure 10 is an elongate, rectangular structure. Also in the illustrated embodiment, the enclosure 10 is capable of interacting with devices for transporting or moving the container or enclosure 10 from one location to another, and such devices may include, for example, hitching means (e.g., a hitch on a vehicle), support structures, and/or wheels. In the currently preferred embodiment, the enclosure 10 includes a floor, walls, a roof, a hitch mechanism (not shown), support structures, and an axle(s) and/or wheels attached thereto. As shown in FIGS. 1-3, the enclosure 10 is a standard semi-trailer or shipping container fitted with the system's equipment and other features. The enclosure 10 may be moved or otherwise transported by means that detachably couple to the enclosure 10, such as a trailer truck via a hitch system, or may have such means permanently coupled thereto. When the enclosure 10 is kept to known or pre-determined dimensions, such as standard semi-trailer dimensions, the system is able to be transported on common roadways and thoroughfares. Also, if kept to standardized container or mobile equipment dimensions, the enclosure 10 is able to utilize shipping docks or other structures previously constructed and designed to accommodate such devices. In the illustrated embodiment, the enclosure 10 is no more than about 9 feet, 6 inches high, in the range of about 30 feet to about 53 feet long, and is not elevated more than about 4 feet from the ground by support/transportation structures, e.g., an axle and wheel assembly. In another embodiment, the enclosure 10 is no more than about 8 feet high. In another exemplary embodiment, the enclosure 10 includes at least one axle positioned approximately under the rear-half of the enclosure, and a hitch at the opposite end of the enclosure. In such an embodiment, the enclosure 10 further includes at least one support structure 62 under the enclosure 10 to support the enclosure 10 and system therein when, for example, the hitch is disengaged from a motor vehicle.

In one exemplary embodiment, the enclosure 10 is shaped and dimensioned to be placed on a carrier platform that is mobile, such as a carrier platform with wheels, as well as on a permanent structural stand and/or directly on ground level. However, those skilled in the art will recognize that many variations of the enclosure 10 are possible, including variations in size, shape, configuration, components and/or compatibility with transportation means, without departing from the spirit and scope of the present invention, including for example structures capable of being transported by wheels, flatbed trailers, trailer trucks, boats, and by railway. Accordingly, the term “containerized” is used herein to mean any enclosure, container or device that contains at least a plurality of the components of the system (e.g., the autoclave and shredder), and that may or may not itself be mobile. For example, in one embodiment, the enclosure is defined by a standard shipping container that can be transported on a flat bed trailer, railcar or boat. In other embodiments, the container itself is mobile, and may be defined by, for example, a trailer that includes wheels and a hitch for hitching the trailer to a semi or tractor-trailer.

As shown in FIGS. 1-3, the enclosure 10 includes a side door 14 located on a side wall at about the midpoint of the enclosure 10. The side door 14 extends from the lower portion of the enclosure 10 and is designed so the lower edge or sill of the side door 14 is about even with the enclosure's floor. The side door 14 may be designed to swing or slide open, mechanically if desired, to expose an opening in the side wall of the enclosure 10 and allow for unabated egress and ingress. In order to protect the sterilization and processing equipment, and for the comfort of workers inside the enclosure 10, the side door 14 may be airtight and/or watertight. The side door 14 and/or opening may or may not extend to the ceiling or roof of the enclosure 10, but should be sized and shaped so as to accommodate an ordinary sized person and for the introduction of the carts 16 of waste into the enclosure 10. As may be recognized by those of ordinary skill in the pertinent art based on the teachings herein, the enclosure 10 may include additional side doors 14 located on the walls of the enclosure, such as side doors located on opposite side walls at about the midpoint of the enclosure 10. Such an arrangement would allow egress and ingress from either side of the enclosure 10. Those skilled in the art will recognize that many variations of the side door 14 are possible, including quantity, size, shape and configuration, without departing from the spirit and scope of the present invention.

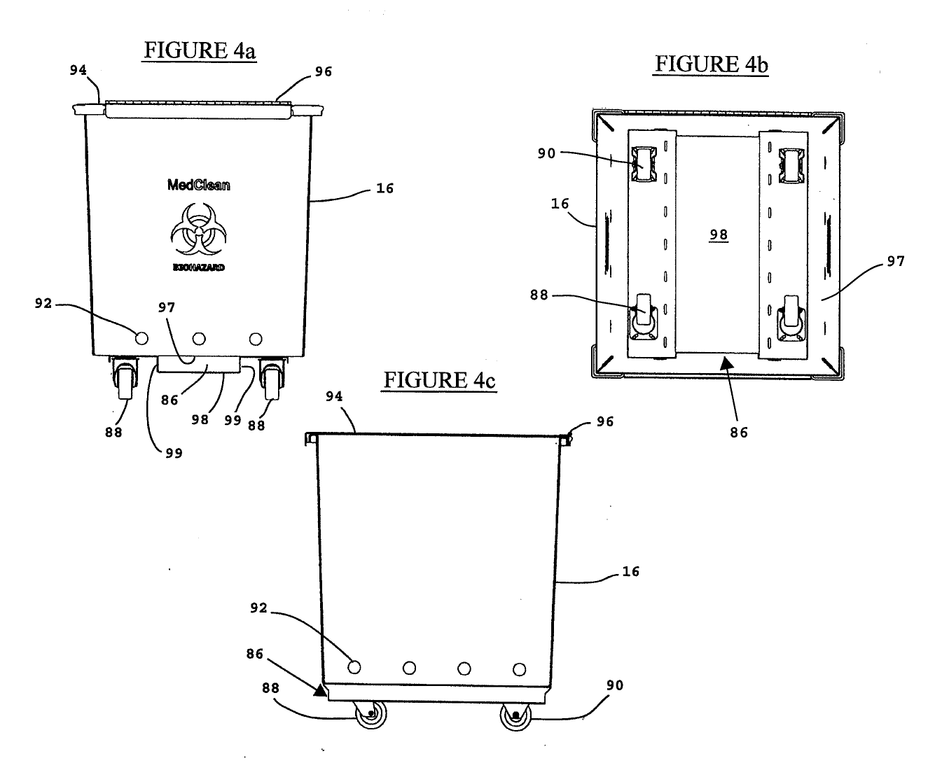

In the illustrative embodiment shown in FIGS. 4a-4c, the carts 16 are capable of holding several cubic feet of waste and are mobile. The carts 16 are rectangular shaped (see FIG. 4b) and taper inwardly from the top to the base 87 of the carts 26 (see FIGS. 4a and 4c). In the illustrative embodiment, the length of the top of the side walls is about 32¾ inches, the length of the front and back walls is about 30¾ inches and the carts 16 are about 36 inches tall. To achieve mobility, the carts 16 include wheels or rollers coupled to the base 97 of the carts 16. In the illustrative embodiment, the carts 16 have wheels or rollers substantially near each corner of the cart's base 97, including two caster or swivel type wheels 88 that allow for turning, and two fixed wheels 90 that are restricted to bidirectional linear movement. In the illustrative embodiment, the carts 16 also include a tipper slot 86 located under the base 97 of the cart 16 and between the wheels or rollers 88, 90. The tipper slot 86 is a rectangular, elongate slot that is open at the front and back sides of the carts 16. The tipper slot 86 is defined by the bottom of the base 97 of the carts 16, a sill surface 98 and slot side walls 99 extending between the base 97 and the sill surface 98. The tipper slot 86 extends linearly from the front to the back of the carts 16, but is recessed from both the front and back side edges of the carts 16. The function of the tipper slot 86 is explained in further detail below in relation to the tipper 24. In the illustrative embodiment, the carts 16 also include a lid 94 coupled to the top of the back wall of the carts 16 by a hinge 96. The lid 94 and hinge 96 are dimensioned and configured so that the lid 94 lays flush with the top of the side walls of the carts 16 in a “closed” position to substantially prevent waste, fumes and the like from escaping the carts 16, such as when, for example, the carts 16 are transported into and about the enclosure 10.

The side walls, front and back walls, base 97 and lid 94 of the carts 16 are made from a material that is durable enough to handle medical waste, such as sharps, and able to withstand the high temperature, pressure and moisture parameters associated with an autoclave sterilization process, such as the sterilization process described in further detail below. The thickness of the material gives the carts 16 sufficient rigidity and sturdiness to securely hold a mass of waste therein. On the other hand, a material that is thinner will transfer heat more quickly through the material and absorb less total heat, so as to decrease the autoclave time and total energy and interfere with sterilization of the waste by an autoclave as little as possible. In addition, the thinner the material, the less the cart weighs, increasing the mobility of the cart and ease of use by a user. In the illustrative embodiment, a 5000 series aluminum alloy, such as Aluminum 5052-H32, with a material thickness of about ⅛ inch thick used. This provides adequate durability and acceptable material thickness and weight with associated heat transfer and specific heat characteristics. Other materials may be used that provide such adequate characteristics. In preferred embodiments, the material has a specific heat capacity of about ⅕ BTU/pounds-degree Fahrenheit and a thermal conductivity of about 960 BTU/pounds-degree Fahrenheit.

The carts 16 also include steam penetration apertures 92 in the side walls of the carts 16. The steam penetration apertures 92 allow steam to more easily enter the interior of the carts 16 during the sterilization process in, for example, the autoclave 18, as compared to a cart that does not include such apertures. In the illustrative embodiment, the steam penetration apertures 92 are located around the periphery of the bottom portion of the side, back and front walls of the carts 16 adjacent the base portion 97. The steam penetration apertures 92 are arranged linearly and are equally spaced from one another along the side, front and back walls of the carts 16. Three steam penetration apertures 92 are formed in the lower portion of the front and back walls, and four steam penetration apertures 92 are formed in the side walls of the carts 16. The steam penetration apertures 92 allow steam to enter the interior of the carts 16 and contact the contents of the carts 16, such as a liner that is positioned in the carts 16 and that contains waste therein.

The carts 16 also may contain a liner (not shown) that is durable enough to handle medical waste, such as sharps, and is able to withstand the high temperature, pressure and moisture parameters associated with autoclave sterilization (e.g., not deteriorate or melt), such as the sterilization process described in further detail below. The also liner prevents liquids and other materials from escaping from the carts 16. The liner also resists sticking, melting, adhering or otherwise becoming coupled to the carts 16 during sterilization in an autoclave. In a preferred embodiment, the liner is made from a polypropylene blend including a slip and an anti-block component, and has a melting point of about 159 degrees Centigrade.

In addition, both the cart material and the liner should be selected so as to prevent the liner from becoming stuck against the cart 16 material surface due to thermal expansion and contraction during the autoclave temperature/pressure cycling. More specifically, it has been observed that with some combinations of liners and cart materials, the liner material becomes entrained in the microscopic surface structures of the cart material, e.g., pores, preventing removal of the liner from the cart surface, e.g., for disposal, or otherwise making removal more difficult. This entrainment is not a function of the liner material melting, deforming or plastically flowing into the surface structures at elevated temperature, or it molecularly bonding to the cart material. Rather, the entrainment seems to be primarily a function of the surface features (hereinafter referred to generally as “pores”) expanding due to heating during autoclaving, the liner material moving into the expanded pore space, and then becoming physically entrained or pinched in the pores when they contract during cooling. The above-discussed aluminum alloy cart materials and polypropylene liner materials do not exhibit excessive sticking in the autoclave processes discussed herein.

When a particular liner will become entrained with a particular cart material depends on a number of factors. One would be the amount of thermal expansion/contraction of the material pores. This, in turn, is dependent upon both the expansion properties of the material, i.e., and coefficient of thermal expansion, and the overall temperature differential obtained during autoclaving. Relatedly, the number of thermal cycles during the autoclaving process may be a factor, as additional expansion/contraction cycles may allow more liner material to become entrained.

Other factors relating to cart material could include the number of pores, the diameter and shape of the pores, the depth of the pores, and the overall surface features, e.g., surface roughness, of the material. For example, the larger the pore, the greater the actual physical expansion during heating. As another example, greater surface roughness provides more surface area with which the liner might become entrained or coupled.

Liner materials characteristics are also relevant. While bulk physical properties of the liner, such as flexibility and bending of the liner at temperature may play a role in potential entrainment, the surface characteristics of the liner are potentially important. A rougher or more uneven surface provides more material area for entrainment. Further, diametrically smaller but higher surface features are more likely to have the size and shape to extend into the expanded pores at temperature. For example, a thin but long or tall surface feature on the liner has more potential to be diametrically small enough to fit into an expanded pore and to extend deeper into that pore, increasing the total liner material area retained and subsequent retention force thereon.

Though it is thermal expansion properties of the cart material that can cause liner entrainment, it should be understood that, within a particular class of materials, thermal expansion characteristics of materials do not greatly vary. For example, while steel and aluminum have significantly different thermal expansion characteristics from a metallurgical standpoint, the thermal expansion characteristics of most commercially-used aluminum and aluminum alloys, such as discussed herein, do not vary nearly as significantly. Further, over the temperature range of the autoclaving process, the total thermal expansion of the cart material is a fraction of a percent. Therefore, the surface characteristics of the cart material and/or the liner material play a much larger role in entrainment than the thermal expansion characteristics of one type of cart material over another. Generally, it is desirable to select cart and/or liner materials that present the lower potential entrainable surface area.

To further enhance the ingress and egress of the carts 16 and personnel with respect to the enclosure 10, the preferred embodiment includes a hinged member or platform 34 located on a side of the enclosure 10 and extending from the rear end of the enclosure 10 to at least a respective side door 14, as best shown by FIG. 1. The hinged member 34 pivots from an open position that is substantially perpendicular to the respective side of the enclosure 10 to a closed position that is substantially parallel with the respective side of the enclosure 10. In the “open” position, the hinged member 34 is wide enough to accommodate carts 16 of waste and/or personnel thereon. Such a hinged configuration allows the hinged member 34 to be stored, when in the “closed” position, in a manner that does not interfere with the transportation of the system 100, on roadways for example. However, when the system 100 arrives at a facility, the hinged member 34 can be “opened” and interact with or abut a loading dock, for example, and allow access to the enclosure 10 therefrom. For reference, the hinged member 34 is shown in the “open” position in FIG. 1. Those skilled in the art will recognize that many variations of the hinged member 34 is possible, including quantity, size, shape and configuration, without departing from the spirit and scope of the present invention, such as the inclusion of hinged members 34 on both sides of the enclosure 10 to allow egress and ingress from side doors 14 located on both sides of the enclosure 10.

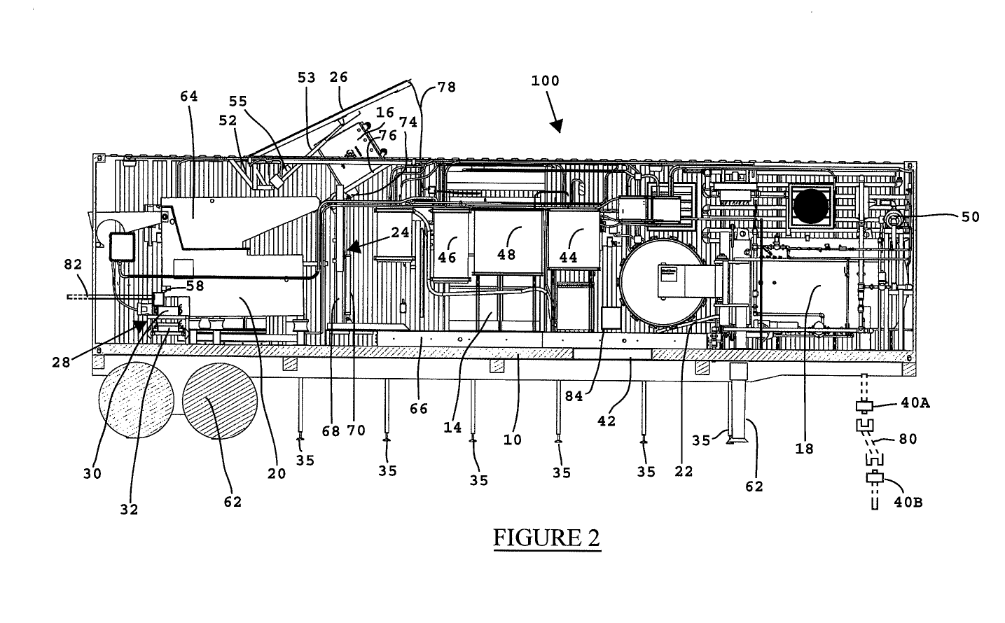

In the illustrative embodiment, the hinge member 34 also includes support structures 35 that extend between the hinge member 34 and the ground to support the hinge member 34 in the “open” position, as best shown in FIG. 2. In consideration of facilities without loading docks or the like, the hinged member 34 includes end portions 36 capable of pivoting downwardly (when the hinged member 34 is in the “open” position) in order to allow the ends of the hinged member 34 to contact the ground. Thereby, the end portions 36 of the hinged member 34 allow ingress and egress to the enclosure 10 by the carts 16 and/or personnel from ground level as well as from an elevated area. In the illustrative embodiment, when the hinged member 34 is in the “open” position, the hinged member 34 includes foldable or portably insertable guard rails 38 along the external peripheries thereof, as best shown in FIG. 1. In such an embodiment, the hinged members 34 are in compliance with regulatory requirements associated with personnel movements within an elevated platform.

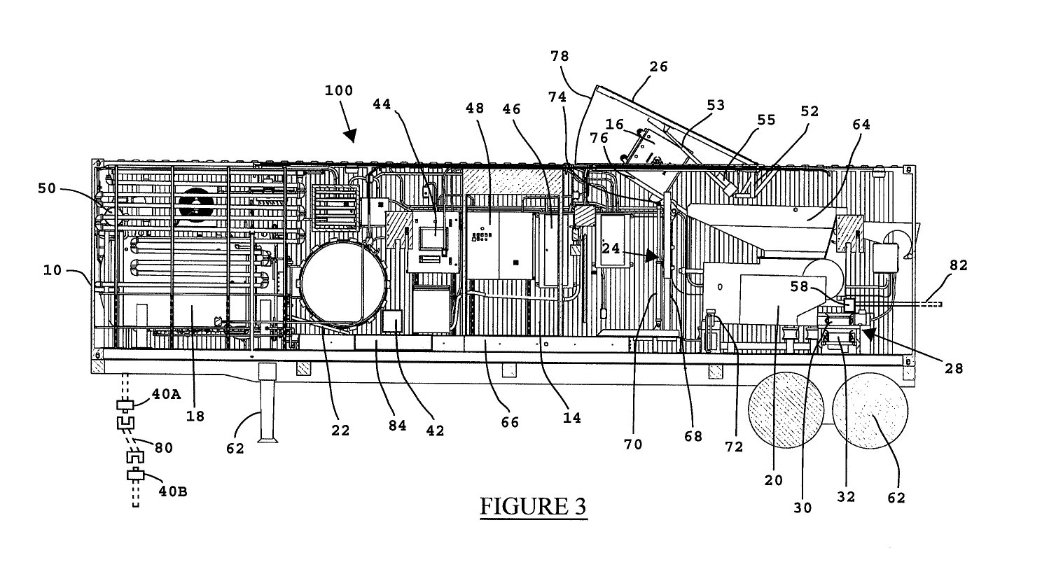

In a preferred embodiment, the waste treatment system 100 further includes an autoclave 18 mounted within the enclosure 10, forwardly of the side door 14, for receiving the carts 16 and the waste therein. In one preferred embodiment, as shown in FIGS. 2 and 3, the autoclave 18 is positioned substantially over a support structure(s) 62 mounted to the underside of the enclosure 10 that interacts with the ground. The containerized treatment system 100 includes the autoclave 18 to sterilize waste through the application of steam—i.e., through the application of heat and pressure. The autoclave 18 includes a substantially airtight treatment chamber to sterilize waste through the application of heat and pressure within the chamber. The autoclave 18 is large enough to contain and sterilize several carts 16 of waste. In some embodiments, the autoclave 18 is capable of holding and sterilizing up to about two (2), and preferably up to about five (5), carts 16 of waste received from the side door(s) 14. The autoclave 18 can be any design or configuration, known or not yet known, but preferably is positioned with the rear of the autoclave 18 facing the front of the enclosure 10. Further, the autoclave 18 should include a sealable opening accessible from an area of the enclosure 10 adjacent to the side door 14 and the shredder 20. Stated differently, the carts 16 of waste should be able to be placed into the treatment chamber of the autoclave 18 through an autoclave sealable door, from the rear of the enclosure 10 and the side door 14. In one embodiment, the treatment chamber of the autoclave is spaced a distance above the floor of the enclosure 10. Therefore, in such an embodiment, a cart track 22 is provided as a means to allow the carts 16 to be positioned inside the autoclave 18. The cart track 22 provides a ramp, extending from the floor of the enclosure 10, such as the an internal floor 66, to the treatment chamber of the autoclave 18, on which the carts 16 of waste can be pushed/rolled over and introduced into the autoclave 18 treatment chamber.

Other equipment associated with the autoclaving sterilization process may be positioned near and/or in contact with the autoclave 18. In the illustrative embodiment, the autoclave 18 makes use of a vacuum pump assembly 50 to apply a vacuum to the carts 16 and waste therein during an autoclaving sterilization process, as described in further detail below. The vacuum assembly 50 is positioned above the autoclave 18 and is in fluid communication therewith. In an alternative embodiment, utilities or equipment associated with the autoclaving process may be located adjacent the autoclave 18 in a separate compartment located forwardly of the autoclave 18. The compartment can be enclosed to insulate the area, for example, because of noise, accessibility, or safety. In such an embodiment, the enclosed area may house a steam generator for the production and supply of steam to the system's equipment, including the autoclave 18, and a water softener and a brine tank for supporting the steam generator. As another example, the compartment may include an electric generator for the production and supply of electrical current to the system's components which utilize electricity, such as a shredder 20.

As shown in FIGS. 1-3, the autoclave 18 and/or associated equipment is controlled and/or analyzed through a control panel 44 located on a wall of the enclosure 10 near the front of the autoclave 18. The control panel 44 can allow a user to modify the autoclave sterilization process to maximize sterilization and efficiency. One variable which may be used to customize the sterilization process through the control panel 44 may be the weight of the waste in the carts 16. In the illustrative embodiment, a scale 42 is sized and positioned in front of the autoclave 18 to determine the weight of the waste entering the autoclave 18 and for customization of the sterilization process. The scale 42 is positioned flush in the floor of the enclosure 10 to allow carts 16 of waste to be easily weighed, information about the carts 16 recorded, and the carts 16 transported over the scale 42 and onto the cart track 22 for entry into the autoclave 18.

A shown in FIG. 2, the scale 42 preferably is set flush into a provided internal floor 66 that is seated or otherwise engaged over the floor of the enclosure 10. The internal floor 66 may also provide a mono-level floor area extending across the internal width of the enclosure 10 (i.e., from one side of the enclosure to the other side of the enclosure) and in the elongated direction from the tipper 24 to the autoclave 18. The mono-level floor area provided by the internal floor 66 allows the carts 16 of waste to be transported throughout the autoclave-side door-tipper area without obstruction. The internal floor 66 also provides an elevated base to reduce the distance between the floor of the enclosure 10 and the entrance of the autoclave 18, thereby reducing the vertical distance that the cart track 22 must span. The decreased vertical component of the cart track 22 creates an easier situation for a user to manually translate a cart 16 up the cart track 22 and into the autoclave 18. In one exemplary embodiment, the internal floor 66 is approximately nine (9) inches in height.

The containerized treatment system further includes a shredder 20, located rearwardly of the autoclave 18 and the side door 14, for processing waste into smaller particles as compared to before processing. As shown in FIGS. 2 and 3, the shredder 20 is positioned substantially over a wheel assembly or support structure 62 mounted to the underside of the enclosure 10 that contacts the ground. In the illustrative embodiment, the shredder 20 is capable of processing waste into unrecognizable pieces as compared to before processing. The waste is processed by the shredder 20 after it is sterilized by the autoclave 18. In the illustrative embodiment, a cart 16 of sterilized waste is removed from the autoclave 18 by manually wheeling the cart 16 out of the autoclave's treatment chamber, down the cart track 22, and across the enclosure's floor (and/or internal floor 66) towards the shredder 20. As is known to those of ordinary skill in the art, the shredder 20 may include a motor, bearings, cutting elements, rotating shafts, a hopper 64, or combinations thereof. As shown in FIGS. 1-3, a shredder electrical box 46 is mounted on or in a wall of the enclosure 10. The shredder electrical box 46 houses the electrical connections that support the shredder 20 and/or mechanisms to control the shredder 20, e.g., on/off switch, speed dials, and/or a safety stop button.

As shown in FIGS. 1-3, the shredder 20 has a hopper 64 for the top loading of material to be introduced into the shredder 20 and processed therein. As can be seen, the hopper 64 may be shaped like a funnel or otherwise shaped and dimensioned to urge or transport contents in the hopper 64 down the hopper 64 and into contact with a shredding means located substantially beneath the hopper 64. The shredder 22 may also include a ram (not shown) positioned at a location to which the hopper 64 feeds waste placed therein. The ram may operate in strokes which push, urge or translate the waste into, or substantially near, a cutting chamber. The cutting chamber is an area in which a rotational cutting mechanism operates. Once waste is in, or substantially near, the cutting chamber, the cutting mechanism may then process the waste in a rotational manner, thereby pulling or otherwise moving the waste from the ram area to a physically spaced output area. A permeable screen may prevent pre-determined sized waste from exiting the cutting chamber and thereby subjecting that waste to further cutting action. Waste that is properly sized to pass through the permeable screen may enter the output area. The output area may include a shoot or other deflection structure designed to deflect or urge the shredded waste into a specific, pre-defined area.

A tipper 24 is provided for receiving a cart 16 of waste from the autoclave 18, and transferring the contents of the cart 16 into the shredder 20 for processing. The tipper 24 includes a base or frame 68 for supporting the tipper's components and a cart 16 of waste thereon. The tipper frame 68 and other components associated with the tipper 24 are preferably positioned relatively close to shredder 20. The tipper frame 68 may be coupled to the floor (or internal floor 66), walls and/or ceiling of the enclosure 10. A vertically extending hydraulic cylinder 70 is coupled to, and thereby supported by, the tipper frame 68. A cart engaging member is coupled to the hydraulic cylinder 70 for vertical movement therewith. In the illustrative embodiment, the cart engaging member is an elongate, flat member which is rotatably coupled to the tipper 24 and extends from the tipper 24 toward the autoclave 18. The cart engaging member is shaped and designed to fit into the tipper slot 86 provided on the underside of the base 97 of each cart 16, as shown best by FIG. 4b and described above.

In the illustrative embodiment, the tipper slot 86 on the underside of the base 97 of cart 16 resembles a ‘U’ shape, thereby providing a substantially enclosed area for the cart engaging member to enter and be supported thereby. The tipper slot 86 may be shaped or designed differently, but should preferably include at least top, bottom and side members to adequately surround the elongate cart engaging member. At a first state (not shown), the cart engaging member is substantially aligned with the tipper slot 86 so that the cart 16 can be manually translated on the floor of the enclosure 10 (or internal floor 66) toward the tipper 24 and the cart engaging member positioned substantially into the tipper slot 86.

At the first state, a hydraulic pump 72 powers the vertical hydraulic cylinder 70 vertically towards the moveable roof panel 26, thereby vertically translating the cart tipper member and the cart 16 thereon. The vertical movement may be initiated by an automatic sensor that recognizes that a cart 16 has been positioned onto the tipper member, or by manually directing (e.g., by pushing a lever) the hydraulic pump 72 to begin powering the vertical hydraulic cylinder 70. At a second state (not shown), the cart 16 is positioned on the cart engaging member and sufficiently elevated, as compared to the shredder 20, for transferring its contents into the shredder 20. In an exemplary embodiment, a sensor may detect sufficient elevation of the cart 16 and stop further vertical movement. In another embodiment, the second state represents the maximum vertical height that the vertical hydraulic cylinder 70 is capable of achieving. At the second state, a second hydraulic cylinder is powered by the hydraulic pump 72 to rotate the cart engaging member, and the cart 16 thereon, about a pivot point 74 towards the roof of the enclosure 10. The second hydraulic cylinder should continue to rotate the cart 16 past horizontal to a third state (as shown in FIGS. 1-3) wherein the cart 16 is in a position sufficient for gravity to act on the contents of the cart 16 and urge the contents out of the cart 16 and into the hopper 64 of the shredder 20.

The cart 16 is prevented from translating or falling into the shredder 20 at the third state due to the interaction of the bottom portion of the tipper slot 86 (as viewed when the cart 16 is on the ground) and the bottom surface of the cart engaging member. Friction between the tipper panel 76 and the cart 16 may further prevent the cart 16 from translating toward the shredder 20. The tipper panel 76 is a substantially flat, elongate panel coupled to the vertical hydraulic cylinder 70 and positioned between the shredder 20 and a cart 16 engaged with the tipper 24. From the perspective of the first state, the tipper panel 76 should be substantially taller and wider than the cart 16. In the third position, as shown in FIGS. 1-3, because the tipper panel 76 is tall and vertically exceeds the pivot point 74, the top of the tipper panel 76 is rotated into a position that abuts, or is located over, the hopper 64 of the shredder 20. As such, the tipper panel 76 acts to guide the contents, both solid and liquid, from the cart 16 into the hopper 64 and therefore into the shredder 20. If the height of the cart 16 does not exceed the pivot point 74 to such a degree that in the third state the top of the cart 16 is located over the hopper 64 of the shredder 20, without the tipper panel 76 the contents of the cart 16 would fall between the hopper 64 of the shredder 20 and the tipper 24.

In one embodiment, the first, second and third states, as referenced above, are automatically controlled through sensors which detect the movements and positions of the tipper's components and the cart 16 thereon. The tipper 24 includes a tipper control box 48 for controlling the vertical movement and rotating or pivoting movement of the tipper 24 and a cart 16 thereon. As shown in FIGS. 1-3, the tipper control box 48 is located on a wall of the enclosure 10. In yet another embodiment, the system 100 may provide for a protective zone around the tipper 24 which, if occupied by a person, automatically stops any movement of the tipper 24. The protective zone may be provided through the use of lasers and/or a cage arrangement positioned near the tipper 24.

The waste treatment system further includes a moveable roof panel 26 for clearance between a cart 16 on the tipper 24 and the roof of the enclosure 10. The moveable roof panel 26 is hinged or otherwise configured to open and create more vertical space above the tipper 24 and/or shredder 20. The moveable roof panel 26 is designed and configured to maintain the integrity of the enclosure 10 in the “open” or “closed” positions of the roof panel 26. To maintain a substantially enclosed area in the open position, a flexible material 78 is coupled between the areas of the moveable roof panel 26 and enclosure 10 that separate from one another as the movable roof panel 26 travels into the “open” position from the “closed” position. The flexible material 78 is folded or otherwise gathered in the closed position and pulled substantially taut in the “open” position. The flexible material 78 can be pre-shaped and dimensioned to the required coverage area in the fully “open” position. In one embodiment, the flexible material 78 is a canvas. The moveable roof panel 26 may be motorized and controlled by a control box, such as the tipper control box 48, or may be mechanically engaged or otherwise linked with the tipper 24 and thereby operate as an automatic step in the tipper's operation. In the illustrative embodiment, as shown in FIGS. 1-3, the moveable roof panel 26 is pivoted between “open” and “closed” positions by hydraulic cylinders 53 pivotally coupled between the roof panel 26 and a support frame 52. The hydraulic cylinders 53 are powered by respective hydraulic pumps 55 fluidly coupled to the hydraulic cylinders 53. As the hydraulic cylinders 53 are extended, the movable roof panel 26 is pivoted into the “open position” and the hydraulic cylinders 53 pivot between the movable roof panel 26 and the support frame 52 accordingly. In one embodiment, the moveable roof panel 26 is maintained in the “open” position to allow multiple carts 16 to be repeatedly tipped over the shredder 20 and then moved to the “closed” position after the multiple-cart processing has finished.

As shown in FIGS. 1-3, after a cart 16 of waste has been sterilized by the autoclave 18, the waste transferred from the cart 16 into the shredder 20 by the tipper 24, and processed into small pieces by the shredder 20, it is directed by the output area of the shredder 20 onto a two-belt conveyor system 28. The two-belt conveyor system 28 allows sterilized, processed waste to be directed away from the enclosure 10 and into a refuse bin or receptacle 60, such as a compactor or Dumpster™. The two-belt conveyor system 28 utilizes two belt conveyors, each including two or more pulleys, with a continuous loop of material (the belt) that rotates about them. In each conveyor, at least one of the pulleys is powered to move the belt, and any material (e.g., waste) on the belt, in the direction dictated by the powered pulley.

One of the belt conveyors of the two-belt conveyor system is an upper main belt 30. The upper main belt 30 is permanently positioned under the outlet of shredder 20 to receive shredded, sterilized waste therefrom. The upper main belt 30 is sized, lengthwise, smaller than the width of the enclosure 10. The upper main belt 30 preferably is automatically activated whenever the shredder 20 is in the process of shredding waste, or conversely, may be individually controlled. In the preferred embodiment, the upper main belt 30 is motorized and able to translate waste thereon laterally across the width of the enclosure 10 (from one side wall to the other side wall). A control box/panel may be incorporated with the two-part conveyor system 28 to control the direction of the upper main belt 30. For example, if the containerized treatment system 100 is positioned along a facility's loading dock, the upper main belt 30 can translate the waste in a direction away from the dock and toward, for example, an adjacent or nearby receptacle 60.

To further enhance the processing and output capability of the containerized treatment system, a preferred embodiment includes a lower translatable belt 32, located beneath the upper main belt 30, as the second part of the two-part conveyor belt system 28. The lower translatable belt 32 is capable of being translated across the width of the unit, and thereby extended from a side of the enclosure 10 a pre-defined distance. In such an embodiment, the lower translatable belt 32 is controlled and powered to translate waste in the same direction as the upper main belt 30. In use, the upper main belt 30 translates waste from the output of the shredder to the edge of the upper main belt 30. Once the waste reaches the edge of upper main belt 30, waste is gravity fed onto the lower translatable belt 32 and further translated away from the enclosure 10 and, preferably, into a receptacle 60 positioned near the enclosure 10 and under the lower translatable belt 32, such as a compactor or Dumpster™. As may be recognized by those of ordinary skill in the pertinent art based on the teachings herein, the particular type of conveyor system described herein is only exemplary, and any of numerous types of conveyor systems that are currently known or that later become known equally may be employed, such as systems with more than two belts, systems with one belt, systems which include belts that can pivot with respect to one another, or other non-belt systems capable of transporting shredded, sterilized waste from the output of the shredder into a nearby receptacle.

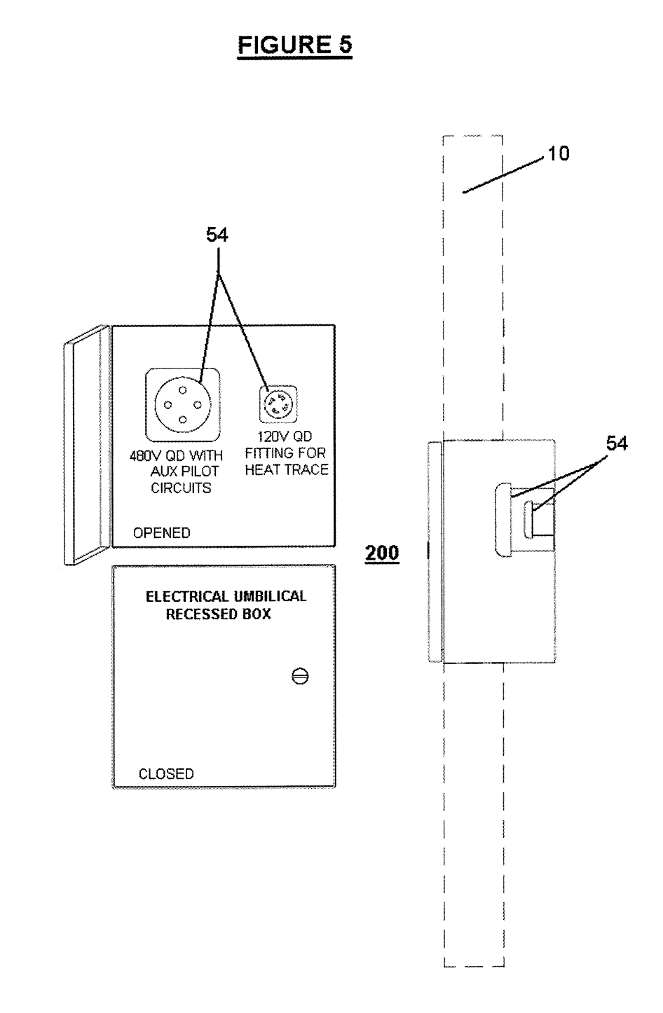

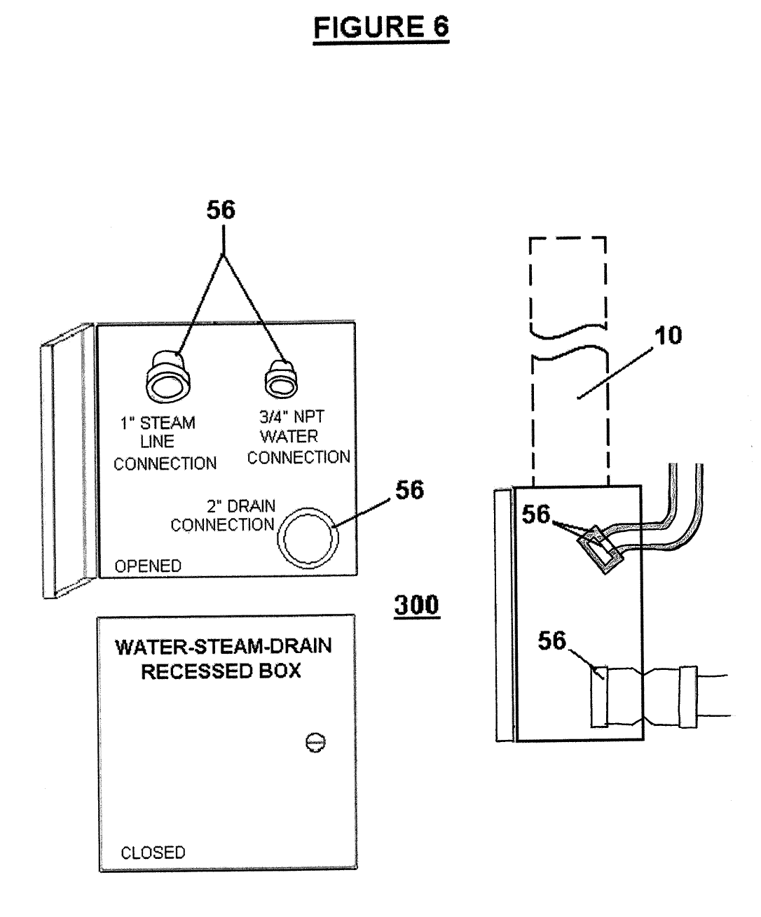

Turning to FIGS. 5 and 6, one preferred embodiment of the system 100 includes at least one utility panel incorporated in a wall or the floor of the enclosure 10 to allow utilities to be provided to the system from an outside source, such as a medical facility loading dock. Preferably, the utility panels and hook-ups are provided in the floor of the enclosure such that they are accessible from underneath the enclosure 10. Such an embodiment protects the utility panels and hook-ups from, for example, inclement weather. The utilities which may be provided to the system 100 include a 480V electrical, 120V electrical, steam, water, and sanitary drain connections. These utility connections may be “quick disconnect” type connections that only require “plug and twist” action to affix and remove the connections, such as the 480V and 120V electrical connections 54 shown in FIG. 5. Other connections, such as the water connection 56 shown in FIG. 6, may utilize a national pipe thread, taper-type connection. As shown in FIGS. 5 and 6, the electrical connections 54 may be housed in their own umbilical box so as to separate the electrical 54 from the water connections 56, due to safety concerns. As may be recognized by the those of ordinary skill in the pertinent art based on the teachings herein, the particular types of connections described herein are only exemplary, and any of numerous types of connections that are currently known or that later become known equally may be employed.

In FIG. 5 an electrical box is indicated by the reference number 200 and includes electrical connections 54 therein. The electrical box may provide for both 480V and 120V electricity. The 480V connection may supply the sterilization and processing equipment, such as the shredder 20, autoclave vacuum pump 50, moveable roof panel 26, side doors 14, conveyor system 28, and the like. The 120V connection may supply other devices, such as interior and exterior lights, control panels, heat tracers to keep water lines from freezing, interior heating and cooling systems, and the like.

In FIG. 6 a non-electrical utility box is indicated by the reference number 300 and includes non-electrical utility connections 56 therein. The non-electrical box may provide steam, water and drain utility connections. The steam connection may supply the sterilization equipment, such as the autoclave 18. The water connection may supply other devices, such as the steam generator 12. The drain connection may provide a sanitary drain, as opposed to a sewer/storm drain, for any sanitary liquid waste used or created by the system, including the autoclave 18. In one embodiment, the drain connection is a pipe or tube leading from the drain connection to a sanitary drain. In some embodiments, the containerized treatment system 100 includes utility connections located at the facility that correspond to the utility connections mounted on the enclosure 10 to provide utilities to the components of the system 100, such as two electrical connections, a steam connection, a water connection, and a drain connection. In some such embodiments, the containerized treatment system 100 further includes an umbilical cord 80 for connecting utility connections on the enclosure 10 to corresponding utility connections at a facility.

In the illustrative embodiment, as shown in FIGS. 2 and 3, the containerized treatment system 100 may include an umbilical connection 40A, extending from the enclosure 10, which houses the utility connections (such as the electrical connections 54 and/or the non-electrical utility connections 56) and is capable of providing utilities to the system 100. Preferably, the system further includes a substantially similar umbilical connection 40B located at a facility that is capable of connecting to the enclosure umbilical connection 40A, and thereby provide utilities to the system 100.

In a currently preferred embodiment of the present disclosure, the containerized treatment system includes a document destruction and collection device. The containerized waste treatment system can be capable of paper document destruction with, for example, the onboard waste shredder 20. The documents are loaded into the shredder 20 similar to sterilized waste, in that documents can be loaded through carts 16 and the tipper 24 associated with the shredder 20. Further, the containerized treatment system may include a collection and transport means to collect and transport the destroyed paper documents from the outlet of the shredder 20. The collection and transport means includes a vacuum system 58 mounted adjacent to the two-belt conveyor system 28 in such a way as to apply a downwardly directed suction to the top of the upper conveyor belt 30, which is sized and positioned to receive shredded waste discharged at the outlet of the shredder 20. In this embodiment, the vacuum system 58 removes shredded documents from the upper conveyor belt 30 and transports the material to a waste receptacle through a conduit 82, such as pipe or tube.

The disclosed systems further includes a computer or computerized controller 84 to take, receive, and/or store data relating to the status of the system, such as but not limited to, the number of carts processed, autoclaving times, the amount of waste processed, the amount of time at a particular position, utility consumption, and conditions inside the enclosure 10. The system may further include the ability to transmit the collected data to, or allow the data to be retrieved from, a remote location, such as by satellite, or other wireless or network devices that are currently known, or that later become known.

The sterilization method of the system 100 may be customized to minimize autoclaving time and, therefore, increase efficiency. One such sterilization method includes the following steps:

(i) Providing an autoclave;

(ii) Placing a load to be sterilized in the autoclave;

(iv) Sealing the autoclave to achieve a substantially air-tight space within the autoclave and surrounding the load;

(v) Performing a first evacuation, wherein the first evacuation removes a pre-determined amount of air from the autoclave and brings about a pre-determined first pressure below atmospheric to the load;

(vi) Introducing a pre-determined amount of condensable vapor having transferable latent heat at a pre-determined first temperature to the substantially air-tight space; This introduction brings about a pre-determined first pressure above atmospheric to the load;

(vii) Allowing the condensable vapor to transfer heat to the load for a first time period under the first pressure above atmospheric;

(viii) Performing a second evacuation, wherein the second evacuation removes a pre-determined amount of the condensable vapor and air from the autoclave to bring about a second pressure below atmospheric to the load; The second pressure below atmospheric is substantially similar to the first pressure below atmospheric;

(ix) Introducing a pre-determined amount of condensable vapor having transferable latent heat at a pre-determined second temperature to the substantially air-tight space; This introduction brings about a pre-determined second pressure above atmospheric to the load; The pre-determined second pressure above atmospheric is substantially similar to the pre-determined first pressure above atmospheric;

(x) Allowing the condensable vapor to transfer heat to the load for a second time period under the second pressure above atmospheric; The second time period is substantially the same as or similar to the first time period;

(xi) Performing a third evacuation, wherein the third evacuation removes a pre-determined amount of the condensable vapor and air from the autoclave to bring about a third pressure below atmospheric to the load; The third pressure below atmospheric is substantially the same as or similar to the first and second pressure below atmospheric;

(xii) Introducing a pre-determined amount of condensable vapor having transferable latent heat at a pre-determined third temperature to the substantially air-tight space; This introduction brings about a pre-determined third pressure above atmospheric to the load; The pre-determined third pressure above atmospheric is substantially similar to the pre-determined first and second pressure above atmospheric;

(xiii) Allowing the condensable vapor to transfer heat to the load for a third time period under the third pressure above atmospheric; The third time period is substantially longer as compared to the first and second time periods to substantially sterilize the load; and

(xiv) Performing a fourth evacuation, wherein the fourth evacuation removes a pre-determined amount of the condensable vapor and air from the autoclave to bring about a fourth pressure below the third pressure.

In the exemplary embodiment, the fourth evacuation may lower the pressure in the autoclave so that the autoclave can be opened without large amounts of steam and pressure escaping. Also, such a feature may allow the autoclave door or cover to be easily opened or the autoclave's substantially airtight portion unsealed. In one embodiment, the sterilization method includes the step of allowing the load to cool to a handling temperature before the autoclave is opened, the airtight portion unsealed, or the load removed from the autoclave.

Preferably, the time periods, temperatures and pressures of each step are pre-determined and specifically designed for a particular load or application. By varying the aforementioned variables, the disclosed autoclaving method can be tailored to achieve sterilization in a shorter time period as compared to previously known methods by removing air from the load. In one preferred embodiment, the first and second time periods are in the range of about 2 minutes to about 10 minutes, the first, second, and third temperatures are in the range of about 275 degrees Fahrenheit to about 300 degrees Fahrenheit, and the first, second, and third pressures below atmospheric range from about 6 inches of water vacuum to about 20 inches of water vacuum. In one embodiment, the third time period is in the range of about 20 minutes to about 45 minutes. In another embodiment, the third time period is at least 1½ times the first and second time periods. In some embodiments, all of the first, second, and third temperatures are sufficiently high to sterilize medical waste. In some embodiments, the step of introducing a pre-determined amount of condensable vapor having transferable latent heat is done rapidly, for example, by injecting the steam into the autoclave to relatively quickly bring about the pre-determined pressure above atmospheric to the load.

In one embodiment, steps (viii), (ix), and (x) define a cycle. In this embodiment, the cycle may be repeated at least one time before step (xi) is formed. The amount of cycles performed on a load can be a variable that can be changed to suit a particular load or application due, for example, to the weight of the load. In one such example, an extremely porous load or a load with large amounts of trapped air within the load may require more cycles than a non-porous load. Other examples of loads which may require the cycle to be repeated at least one time, are loads which include air pockets in the space within a cylindrical member, such as a syringe, or form that air pockets in difficult-to-access areas of the load. Another example of loads which may require the cycle to be repeated at least one time are loads with errors in packaging, such as overloading of the sterilizer chamber of the autoclave.

As previously discussed, the exemplary sterilization methods of the present disclosure may reduce the sterilization procedure time as compared to previous methods because of the removal of air pockets. As such, in the exemplary embodiments, the load may form at least one air pocket within the mass of the load during the method, which is then removed from the load and autoclave during the method. Without being limited to a particular theory, the disclosed methods may advantageously remove air pockets from the load being sterilized because the vacuum acts to give the load buoyancy as compared to a load that is not subjected to a vacuum. Therefore, the vacuum may act to prevent the load from bearing down upon itself and thereby forming air pockets. The buoyancy feature and the introduction of steam may allow the steam to penetrate the load advantageously and remove the air pockets as compared to the load without a vacuum. The method may be characterized as not allowing the load to bear down upon itself and thereby form air pockets which cannot be penetrated by steam and the load thereby insulates itself against the high temperatures of the steam. The method may be further characterized as a loosening of the load to allow the air pockets to be removed from the load, which allows for steam to penetrate the entire mass of the load and prevent the load from insulating itself against the high temperatures of the steam.

It may be readily understood by those having skill in the pertinent art from the present disclosure that any of numerous changes and modifications may be made to the above described and other embodiments of the present disclosure without departing from the scope of the invention as defined in the appended claims. For example, the containerized medical treatment system may be made from any of numerous different materials, in any of numerous shapes, taking any of numerous different dimensions. In addition, the utility connections may be releasably attachable to the containerized unit in any of numerous different ways that are currently known, or that later become known. The term “enclosure” is used herein to broadly define any transportable structure capable of housing in whole or in part sterilization or processing equipment, and may or may not include walls, a roof, support stilts, hitch means, axles, wheels, etc. The phrase “autoclave” is used herein to mean a pressurized device designed to heat aqueous solutions above their boiling point at normal atmospheric pressure to achieve sterilization. The term “shredder” is used herein to mean any means capable of breaking apart or processing a load material of one size into relatively smaller pieces, and may or may not include a rotating shaft, motor, cutting element, gears, hopper, bearings, and control means. The term “tipper” is used herein to mean any means capable of accepting a cart or like device for holding a load of waste and transferring the load into a shredder, including without limitation a hydraulic lifting and tipping mechanism. Accordingly, this detailed description of the currently preferred embodiments of the present disclosure is to be taken in an illustrative, as opposed to a limiting sense.

What is claimed is:

1. A containerized waste treatment system for sterilizing and processing waste, the system comprising:
an elongate enclosure defining a front end, a rear end, at least two side walls laterally spaced on substantially opposite sides of the enclosure relative to each other and extending between the front and rear ends of the enclosure, a platform extending in an elongate direction between the front and rear ends and extending laterally between the two side walls of the enclosure, and an access opening extending through at least one side wall of the enclosure that allows entry of an operator and cart into the enclosure and egress out of the enclosure therethrough;
an autoclave mounted within the enclosure on the platform adjacent to either the front end or the rear end of the enclosure;
a shredder mounted within the enclosure on the platform adjacent to either the front end or the rear end of the enclosure, wherein the shredder and the autoclave are spaced relative to each other and define a cart path therebetween within the enclosure, and wherein the cart path is in communication with the access opening to allow the operator to move the cart containing a load of waste through the access opening, into the cart path, from the cart path into the autoclave, sterilize the load of waste in the autoclave, retrieve the load of waste from the autoclave, move the cart with the sterilized load of waste along the cart path to the shredder, move the load of sterilized waste from the cart into the shredder, shred the sterilized waste in the shredder, and dispose of the sterilized, shredded waste.

2. The containerized waste treatment system according to claim 1, further comprising a cart configured for movement through the access opening and along the cart path between the autoclave and shredder.

3. The containerized waste treatment system according to claim 2, wherein the cart includes a liner, and wherein the cart is configured such that the liner does not become coupled to the cart during sterilization in the autoclave.

4. The containerized waste treatment system according to claim 3, wherein the cart is made from a metal material that is at least about ⅛ inch thick and that includes surface features that prevent the liner from becoming coupled to the material after the material expands an contracts during sterilization in the autoclave.

5. The containerized waste treatment system according to claim 4, wherein the surface features are selected from at least one of the number of pores, the amount of thermal expansion and contraction of pores, the diameter of pores, the shape of pores, the depth of pores and the surface roughness of the material.

6. The containerized waste treatment system according to claim 4, wherein the metal material is a 5000 series aluminum alloy.

7. The containerized waste treatment system according to claim 2, wherein the cart contains at least one aperture that allows steam to pass through the at least one aperture and into the interior of the cart during sterilization in the autoclave.

8. The containerized waste treatment system according to claim 1, wherein the access opening is spaced between autoclave and shredder.

9. The containerized waste treatment system according to claim 1, wherein the access opening defines an opening dimensioned such that an ordinary sized person can pass therethrough.

10. The containerized waste treatment system according to claim 1, wherein the enclosure includes a shredded waste outlet in communication between the shredder and the exterior of the enclosure for passage of shredded waste therethrough.

11. The containerized waste treatment system according to claim 1, further comprising a conveyor mounted adjacent to the shredded waste outlet for receiving the shredded waste and transporting the shredded waste on the conveyor away from the enclosure.

12. The containerized waste treatment system according to claim 11, wherein the conveyor includes a first conveyor portion and a second conveyor portion, wherein at least one of the first and second conveyor portions is laterally movable relative to the other between retracted and extended positions.

13. The containerized waste treatment system according to claim 12, further comprising a refuse container near the enclosure, wherein the second conveyor portion is movable between a retracted position in which the second conveyor portion does not extend laterally beyond a respective side wall of the enclosure, and an extended position in which the second conveyor portion extends laterally relative to the respective side wall of the enclosure, and the first and second conveyor portions extend between the shredded waste outlet and the refuse container for transporting shredded waste from the shredded waste outlet to the refuse container.

14. The containerized waste treatment system according to claim 13, wherein the second conveyor portion is movable laterally between retracted and extended positions on opposite sides of the enclosure relative to each other.

15. The containerized waste treatment system according to claim 1, further comprising a front support located beneath the enclosure adjacent to the front end thereof, and a rear support located beneath the enclosure adjacent to the rear end thereof, and wherein one of the autoclave and shredder is mounted approximately over the front support, and the other of the autoclave and the shredder is mounted approximately over the rear support.

16. The containerized waste treatment system according to claim 15, wherein at least one of front and rear supports includes wheels.

17. The containerized waste treatment system according to claim 1, wherein the enclosure is one of a trailer or container mountable on a flat bed trailer.

18. The containerized waste treatment system according to claim 1, wherein the enclosure defines a height from ground level of less than about 13 feet and 6 inches, and a length of less than about 53 feet.

19. The containerized waste treatment system according to claim 1, further comprising a tipper mounted adjacent to the shredder and engageable with a cart containing a load of waste, wherein the tipper is movable between a first position engaging the cart and maintaining the load of waste within the cart, and a second position engaging the cart and tipping the cart to move the load of waste from the cart into the shredder.

20. The containerized waste treatment system according to claim 19, wherein the second position is elevated with respect to the first position.

21. The containerized waste treatment system according to claim 20, wherein the enclosure includes a roof, and the roof includes a portion overlying at least one of the shredder and the tipper and movable substantially vertically relative thereto between a lower position and an upper position, wherein in the upper position the movable roof portion defines additional clearance between the roof and at least one of the tipper and the shredder in comparison to lower position for receiving at least a portion of the cart in the upper position therein.

22. The containerized waste treatment system according to claim 1, further comprising a side platform mounted on the enclosure and movable between (i) a first position wherein the side platform extends laterally outwardly relative to a respective side wall of the enclosure and defines a cart path extending between the rear end of the enclosure and the access opening that allows movement of the operator and cart thereon, and (ii) a second wherein the side platform is located laterally inwardly relative to the first position and does not define the cart path extending between the rear end of the enclosure and the access opening.

23. The containerized waste treatment system according to claim 22, wherein the side platform is pivotally mounted on the enclosure, and in the first position is pivoted inwardly against or adjacent to a respective side wall of the enclosure, and in the second position is pivoted outwardly relative to the respective side wall of the enclosure.

24. The containerized waste treatment system according to claim 22, further comprising two side platforms located on opposite sides of the enclosure relative to each other.

25. The containerized waste treatment system according to claim 22, wherein the platform includes a rearward portion and a forward portion, the rearward portion extends between the rear end of the enclosure and the forward portion, and the forward portion extends between the rearward portion and the access opening, and a rear end of the rearward portion is movable vertically between (i) a lower position allowing access to the panel from ground level, and (ii) an upper position allowing access to the panel from a loading dock level.

26. A containerized waste treatment system for sterilizing and processing waste, the system comprising:
(i) an elongate enclosure including two side walls, a front wall, a back wall, a roof, and a floor;
(ii) at least one side door located on a side wall of the enclosure for introducing a cart of waste into the enclosure;
(iii) an autoclave positioned within the enclosure forwardly of the side door for receiving the waste from the cart and sterilizing the waste by the application of steam;
(iv) a shredder located rearwardly of the at least one side door for shredding sterilized waste;
(v) a cart path extending between the autoclave and the shredder;
(vi) a tipper located forwardly of the shredder and rearwardly of the at least one side door for transferring the contents of the cart into the shredder;
(vii) a moveable roof panel located in the roof of the enclosure above at least one of the tipper and the shredder to allow for increased vertical space and thereby accommodate the cart when lifted by the tipper into a position capable of transferring waste from the cart into the shredder;
(viii) a conveyor positioned to accept shredded, sterilized waste from an outlet of the shredder, and to transport the shredded, sterilized waste;
(ix) a side platform extending along one side of the enclosure between the rear end of the enclosure and the at least one side door, and movable between a (i) use position extending laterally outwardly relative to the respective side of the enclosure and defining a cart path thereon for transporting the cart of waste thereon and through the at least one side door, and (ii) a non-use position located laterally inwardly relative to the use position; and
(xi) utility connections mounted on the enclosure to provide utilities to the components of system, and including at least a plurality of an electrical connection, a steam connection, a water connection, and a drain connection.

27. The containerized waste treatment system according to claim 26, further comprising a cart configured for movement through the access opening and between the autoclave and shredder.

28. The containerized waste treatment system according to claim 27, wherein the cart includes a liner, and wherein the cart is configured such that the liner does not become coupled to the cart during sterilization in the autoclave.

29. The containerized waste treatment system according to claim 28, wherein the cart is made from a metal material that is at least about ⅛ inch thick and that includes surface features that prevent the liner from becoming coupled to the material after the material expands an contracts during sterilization in the autoclave.

30. The containerized waste treatment system according to claim 29, wherein the surface features are selected from at least one of the number of pores, the amount of thermal expansion and contraction of pores, the diameter of pores, the shape of pores, the depth of pores and the surface roughness of the material.

31. The containerized waste treatment system according to claim 29, wherein the metal material is a 5000 series aluminum alloy.

32. The containerized waste treatment system according to claim 27, wherein the cart contains several apertures that allow steam to pass through the apertures and into the interior of the cart during sterilization in the autoclave.

33. The containerized waste treatment system according to claim 26, wherein the conveyor includes a two part conveyor belt, including a lower translatable belt, that is laterally translatable from one side of the enclosure to the other, and an upper stationary belt located at the outlet of the shredder and extending from approximately one side of the enclosure to the other side.

34. The containerized waste treatment system according to claim 33, further including a vacuum assembly mounted adjacent to the upper stationary belt, wherein the vacuum assembly removes shredded paper from the upper stationary belt and transports said shredded paper to a receptacle.

35. The containerized waste treatment system according to claim 33, wherein the two-belt conveyor system transfers the shredded, sterilized waste into a pre-positioned compactor or dumpster.

36. The containerized waste treatment system according to claim 26, wherein the side platform includes a hinged platform located on at least one side of the enclosure, and extending from the rear end of the enclosure to a respective side door, wherein the hinged platform is pivotable between an open position that is substantially perpendicular to the respective side wall of the enclosure and a closed position that is substantially parallel with the respective side wall of the enclosure, and wherein a rear end of the platform is pivotable downwardly toward the ground.

37. The containerized waste treatment system according to claim 36, wherein the hinged member is sufficiently wide to accommodate the ingress and egress of carts and people to the enclosure through the at least one side door.

38. The containerized waste treatment system according to claim 26, further including at least a plurality of an electric generator, steam generator, water softener, brine tank, vacuum pump in fluid connectivity to the autoclave, and combinations thereof.

39. The containerized waste treatment system according to claim 38, wherein the electric generator, water softener and brine tank are located in an enclosed compartment forwardly positioned with respect to the autoclave.

40. The containerized waste treatment system according to claim 26, further including rear wheels mounted to the enclosure or to a platform located beneath the enclosure.

41. The containerized waste treatment system according to claim 40, wherein the rear wheels are located approximately under the shredder.

42. The containerized waste treatment system according to claim 26, further including at least one support structure mounted to the enclosure or to a platform located beneath the enclosure and capable of contacting the ground.

43. The containerized waste treatment system according to claim 42, wherein the at least one support structure is located approximately under the autoclave.

44. The containerized waste treatment system according to claim 26, further including at least a plurality of the following: interior lights, exterior lights, a control panel, heat tracers to keep the water lines from freezing, interior heating system, interior cooling system, and combinations thereof.

45. The containerized waste treatment system according to claim 26, wherein the enclosure is a standard semi-trailer or shipping container.

46. The containerized waste treatment system according to claim 26, wherein the autoclave is substantially airtight, and wherein the application of steam increases the temperature and pressure inside the autoclave.

47. The containerized waste treatment system according to claim 26, wherein the autoclave is capable of receiving therein and substantially simultaneously sterilizing two to five carts of medical waste.

48. The containerized waste treatment system according to claim 26, wherein the tipper includes a base for engaging the cart, a hydraulic cylinder for elevating the cart, and a tipper for transferring the contents of the cart into the shredder.

49. The containerized waste treatment system according to claim 48, wherein the system further sterilizes the waste in the autoclave, shreds the waste in the shredder, and the two-belt conveyor system deposits the waste in the trash receptacle.

50. The containerized waste treatment system according to claim 26, further including a scale positioned within the floor of the enclosure for determining the weight of a cart as it passes over the scale and onto the cart path.

51. The containerized waste treatment system according to claim 26, wherein the shredder is capable of shredding medical waste into unrecognizable pieces as compared to the waste before shredding.

52. The containerized waste treatment system according to claim 26, wherein the system is positioned near a facility and a trash receptacle, and wherein the enclosure receives carts of waste from the hinged member through a side door.

53. A containerized waste treatment system for sterilizing and processing waste, the system comprising:
(i) first means for enclosing and transporting the system;
(ii) second means for accessing the interior of the first means with a cart of waste;
(iii) third means located within the first means for sterilizing the waste;
(iv) fourth means for transporting carts of waste within the first means;
(v) fifth means located rearwardly of the second means within the first means for shredding the waste; and
(vi) sixth means located forwardly of the fifth means within the first means for transferring the contents of the cart into the fifth means.

54. The system according to claim 51, further comprising:
(vii) seventh means for increasing the vertical space over at least one of the fifth and sixth means;
(viii) eighth means positioned to accept shredded, sterilized waste from the fifth means, and to transport the shredded, sterilized waste;
(ix) ninth means for providing access to the first means through the second means from an elevated position or ground level; and
(xi) tenth means for providing utilities to the components of system.

55. The system according to claim 53, wherein the first means is an elongate, substantially rectangular container with two side walls, a front wall, a back wall, a roof, a floor.

56. The system according to claim 53, wherein the second means includes at least one side door located on the first means.

57. The system according to claim 53, wherein the third means is an autoclave mounted on the first means forwardly of the second means for receiving the waste from the cart and sterilizing the waste by the application of steam.

58. The system according to claim 53, wherein the fourth means is a cart path extending between the second, third and fifth means.

59. The system according to claim 53, wherein the fifth means is a shredder.

60. The system according to claim 53, wherein the sixth means is a tipper.

61. The system according to claim 54, wherein the seventh means is a moveable roof panel.

62. The system according to claim 54, wherein the eighth means is a two part conveyor belt.

63. The system according to claim 54, wherein the ninth means is a hinged member located on at least one side of the first means and extends from the rear of the first means to at least the second means, wherein the hinged member can pivot from an open position that is substantially perpendicular to the respective side of the first means, to a closed position that is substantially parallel with the respective side of the first means, and wherein each end of the members are capable of pivoting downwardly in order to contact the ground.

64. The system according to claim 54, wherein the tenth means is utility connections.

65. A sterilization method comprising the following steps:
(i) providing an enclosure including a side door, an autoclave mounted to one side of the side door within the interior of the enclosure, and a shredder mounted to an opposite side of the side door within the interior of the enclosure, and a cart path extending between the side door, autoclave and shredder;
(ii) transporting the enclosure with a vehicle from a first location to a second location;
(iii) with the enclosure located in the second location, introducing a cart containing a load of waste through the side door into the enclosure;
(iv) placing the cart within the autoclave and sterilizing the load of waste within the autoclave;
(v) moving the cart with the sterilized load of waste along the cart path from the autoclave to the shredder;
(vi) moving the sterilized waste from the cart into the shredder and shredding the sterilized waste; and
(vii) discharging the shredded sterilized waste from the enclosure into a refuse container.

66. A method as defined in claim 65, wherein the sterilizing step includes the following:
(i) sealing the autoclave to achieve a substantially air-tight space within the autoclave and surrounding the load;
(ii) performing a first evacuation, wherein the first evacuation removes air from the autoclave and brings about a pre-determined first pressure below atmospheric to the load;
(iii) introducing condensable vapor having transferable latent heat at a pre-determined first temperature to the substantially air-tight space, wherein the introduction brings about a pre-determined first pressure above atmospheric to the load;
(iv) allowing the condensable vapor to transfer heat to the load for a first time period under the first pressure above atmospheric;
(v) performing a second evacuation, wherein the second evacuation removes a pre-determined amount of the condensable vapor and air from the autoclave to bring about a second pressure below atmospheric to the load;
(vi) introducing condensable vapor having transferable latent heat at a pre-determined second temperature to the substantially air-tight space, wherein the introduction brings about a pre-determined second pressure above atmospheric to the load;
(vii) allowing the condensable vapor to transfer heat to the load for a second time period under the second pressure above atmospheric, wherein the second time period is substantially the same as or similar to the first time period;
(viii) performing a third evacuation, wherein the third evacuation removes condensable vapor and air from the autoclave to bring about a third pressure below atmospheric to the load;
(ix) introducing condensable vapor having transferable latent heat at a pre-determined third temperature to the substantially air-tight space, wherein the introduction brings about a pre-determined third pressure above atmospheric to the load;
(x) allowing the condensable vapor to transfer heat to the load for a third time period under the third pressure above atmospheric, wherein the third time period is substantially longer as compared to the first and second time periods, to substantially sterilize the load; and
(xi) performing a fourth evacuation, wherein the fourth evacuation removes condensable vapor and air from the autoclave to bring about a fourth pressure below the third pressure.

67. A method as defined in claim 66, wherein the second pressure below atmospheric is substantially similar to the first pressure below atmospheric; the pre-determined second pressure above atmospheric is substantially similar to the pre-determined first pressure above atmospheric; the third pressure below atmospheric is substantially the same as or similar to the first and second pressures below atmospheric; and the pre-determined third pressure above atmospheric is substantially similar to the pre-determined first and second pressures above atmospheric.

68. A method as defined in claim 66, further comprising the step of allowing the load to cool to a handling temperature after step (xi).

69. A method as defined in claim 66, wherein the first and second time periods are in the range of about 2 minutes to about 10 minutes.

70. A method as defined in claim 66, wherein the third time period is at least one and one half times the first and second time periods.

71. A method as defined in claim 66, wherein the third temperature is sufficiently high enough to sterilize medical waste.

72. A method as defined in claim 66, wherein the first, second, or third temperatures are sufficiently high enough to sterilize medical waste.

73. A method as defined in claim 66, wherein the first, second, or third temperatures are in the range of about 275 degrees Fahrenheit to about 300 degrees Fahrenheit.

74. A method as defined in claim 66, wherein the first, second, and third pressures below atmospheric are within the range of about 6 inches of water vacuum to about 20 inches of water vacuum.

75. A method as defined in claim 66, further including repeating steps (v), (vi) and (vii) at least one time before step (viii).

76. A method as defined in claim 65, wherein the load forms at least one air pocket within the mass of the load, and the at least one air pocket within the mass of the load is removed from the load during the method.

77. A method as defined in claim 65, wherein the fourth pressure allows the autoclave to be safely opened by an operator.

78. A sterilization method comprising the following steps:
(i) sealing an autoclave to achieve a substantially air-tight space within the autoclave and surrounding the load;
(ii) performing a first evacuation, wherein the first evacuation removes air from the autoclave and brings about a pre-determined first pressure below atmospheric to the load;
(iii) introducing condensable vapor having transferable latent heat at a pre-determined first temperature to the substantially air-tight space, wherein the introduction brings about a pre-determined first pressure above atmospheric to the load;
(iv) allowing the condensable vapor to transfer heat to the load for a first time period under the first pressure above atmospheric;
(v) performing a second evacuation, wherein the second evacuation removes a pre-determined amount of the condensable vapor and air from the autoclave to bring about a second pressure below atmospheric to the load;
(vi) introducing condensable vapor having transferable latent heat at a pre-determined second temperature to the substantially air-tight space, wherein the introduction brings about a pre-determined second pressure above atmospheric to the load;
(vii) allowing the condensable vapor to transfer heat to the load for a second time period under the first pressure above atmospheric, wherein the second time period is substantially the same as or similar to the first time period;
(viii) performing a third evacuation, wherein the third evacuation removes condensable vapor and air from the autoclave to bring about a third pressure below atmospheric to the load;
(ix) introducing condensable vapor having transferable latent heat at a pre-determined third temperature to the substantially air-tight space, wherein the introduction brings about a pre-determined third pressure above atmospheric to the load;
(x) allowing the condensable vapor to transfer heat to the load for a third time period under the third pressure above atmospheric, wherein the third time period is substantially longer as compared to the first and second time periods, to substantially sterilize the load; and
(xi) performing a fourth evacuation, wherein the fourth evacuation removes condensable vapor and air from the autoclave to bring about a fourth pressure below the third pressure.

79. A method as defined in claim 78, wherein the second pressure below atmospheric is substantially similar to the first pressure below atmospheric; the pre-determined second pressure above atmospheric is substantially similar to the pre-determined first pressure above atmospheric; the third pressure below atmospheric is substantially the same as or similar to the first and second pressures below atmospheric; and the pre-determined third pressure above atmospheric is substantially similar to the pre-determined first and second pressures above atmospheric.
